(19) United States
(12) Patent Application Publication
(10) Pub. No.: US 2009/0022770 A1
(43) Pub. Date: Jan. 22, 2009

(54) Chitosan Compositions

(76) Inventor: Mats Andersson, Uttran (SE)

Correspondence Address: MCDERMOTT WILL & EMERY LLP, 600 13TH STREET, N.W., WASHINGTON, DC, 20005-3096, US

(21) Appl. No.: 11/793,625

(22) PCT Filed: Dec. 20, 2005
(86) PCT No.: PCT/IB2005/004023

§ 371 Date: May 15, 2008

Related U.S. Application Data
(60) Provisional application No. 60/637,980, filed on Dec. 20, 2004.

(51) Int. Cl. A61F 2/28; A61K 31/722; A61P 19/00; C08B 37/08

(52) U.S. Cl. 424/423; 514/55; 536/20

(57) ABSTRACT

This invention relates to an orthopaedic composition comprising porous chitosan particles suspended in a liquid medium wherein the liquid medium further comprises a biocompatible polymer. The invention also provides a process for preparing a solid or semi-solid orthopaedic material by drying the orthopaedic composition. The resulting solid or semi-solid orthopaedic material finds use as a bone-replacement material, a bone cement and a tissue scaffold. A process for preparing suitable porous chitosan particles for the present invention by incorporating a porogen capable of including crystallinity is also described.

The present invention relates chitosan compositions, and in particular compositions for orthopaedic applications.

Bone replacements are used in a variety of indications, such as, fractures repair, implants revisions, filling of voids after tumours and cysts removal and within spinal indications. An elderly, still active population strongly contributes to the increasing number of surgical procedures requiring bone substitutes. Several years back, the orthopaedic surgeon used the patient's own bone (autograft) in the majority of grafting procedures but nowadays the professionals rely more and more on cadaver bone (allograft) which is available from commercial bone banks or recovered in the hospitals. The dependence on allograft has two weaknesses. First, there is a risk, however small, of viral contamination and costly test procedures have to be used to guarantee patient security. Second, the demand for allogenic products exceeds the supply. Taken together these factors have opened the gates for synthetic bone replacement materials.

Another related area is fracture fixation devices. Metal plates, screws, nails, wires, pins, rods are used for fixation of bone. The fixation has to be somewhat rigid in order to heal the fracture but a too rigid fixation can prevent completion of healing because there is a mismatch between the elasticity of the fixation device and bone. Fixation devices made from stainless steel and titanium have considerably higher Young's modulus compared to bone. Normally these metal implants will stay in the body after healing however sometimes they cause pain and discomfort for the patient and have to be removed in a secondary surgery procedure. In order to reduce the mismatch in rigidity between device and bone polymers like bone cement are used. To further level out these mismatches a number of new materials have been designed.

A third area in which new and better materials and treatment procedures are of interest is in cartilage repair. A vast number of approaches have been tested but so far with limited success. Transfer of living cells and new scaffolds based on many different materials have been studied and the research is very intense. The primary cells of interest in cartilage repair are chondrocytes, which have been seeded either as such or in a scaffold into the damaged area. Examples of scaffolds for chondrocytes are e.g. hyaluronic acid and chitosan.

In both the area of bone filling, bone fixation and cartilage repair extensive research and material development is ongoing. In the bone filling area there are mainly three categories of materials, inorganic ceramic-like materials, synthetic polymers and various mixtures in which some contain allograft. See for example U.S. Pat. No. 6,376,573, U.S. Pat. No. 6,458,375, U.S. Pat. No. 6,696,073, U.S. Pat. No. 6,767,369, U.S. Pat. No. 6,793,725, U.S. Pat. No. 6,372,257, WO 02/080992, US 2002/032488, KR 2001/103306, US 2003/124172, DE 19724869, WO 99/47186, U.S. Pat. No. 6,378,527, U.S. Pat. No. 5,624,463 and WO 03/008007. The cultivation of bone forming cells, osteoblasts, on chitosan scaffolds is described in WO 01/46266 and Macromol. Biosci. 2004, 4, 811-819. WO 01/46266 discloses chitosan beads in the form of a loosely-linked network of chitosan and the article from Macromol. Biosci. Describes chitosan fibres.

Hydroxyapatite is used in a number of different compositions. It is biocompatible, osteoconductive, non-toxic and non-immunogenic. Particulate hydroxyapatite is however unstable when mixed with the patient's blood and can migrate to surrounding tissue. Calcium phosphate cement can conform to cavity shapes and harden in situ to form solid hydroxyapatite. The potential advantage offered by a porous ceramic implant is its inertness combined with the mechanical stability of the highly convoluted interface that develops when bone grows into the pores of the ceramic. The microstructure of certain corals makes an almost ideal material for obtaining structures with highly controlled pore sizes. Corals have been found suitable in some orthopaedic applications where the mechanical requirements are of less importance since coral is considered to be brittle and lack tensile strength.

In bone filling a huge number of organic polymers have been tested. Both naturally occurring materials like proteins, e.g. collagen and polysaccharides e.g. hyaluronic acid, chitosan, chitin and synthetic polymers e.g. polylactides and polyglycolides have been used.

Mixtures of inorganic and organic materials are used in a vast number of applications often in combination with demineralised bone. Depending on the area of use the materials are given specific properties with regard to hardness, biodegradability and porosity. Additives like different growth factors, bone morphogenic protein to stimulate further bone formation, and anti-bacterial agents are also common in these mixtures.

In bone fixation devices where the mechanical properties are of utmost importance synthetic materials based on biodegradable polymers of lactic or glycolic acid are the most frequently used and several products from these are now found on the market. PLA and PGA and copolymers thereof have been investigated for more applications than any other degradable polymer. The interest in these materials is based, not on their superior materials properties, but primarily on the fact that these polymers have already been used successfully in a number of approved medical implants and are considered safe, biocompatible, and non-toxic by regulatory agencies in virtually all developed countries. Therefore, implantable devices prepared from PLA, PGA, or copolymers thereof can be brought to market in less time and for a lower cost than similar devices prepared from novel polymers whose biocompatibility is still unproven. Currently available and approved products include sutures, GTR membranes for dentistry, bone pins, and implantable drug-delivery systems. The polymers are also being widely investigated in the design of vascular and urological stents and skin substitutes, and as scaffolds for tissue engineering and tissue reconstruction. In many of these applications PLA, PGA, and copolymers thereof have performed with moderate to high degrees of success. However, there are still unresolved issues: First, in tissue culture experiments, most cells do not attach to PLA or PGA surfaces and do not grow as vigorously as on the surface of other materials, indicating that these polymers are actually poor substrates for cell growth in vitro. Second the degradation products of PLA and PGA are relatively strong acids (lactic acid and glycolic acid). When these degradation products accumulate at the implant site a delayed inflammatory response is often observed months to years after implantation.

After a device has been implanted, adsorption and absorption process occur, polymeric surfaces in contact with body fluids immediately adsorb, proteinaceous components, and the bulk begins to absorb soluble components such as water, proteins and lipids. Cellular elements subsequently attach to the surfaces and initiate chemical processes. With biocompatible materials, the foreign body reaction in the implant site may be controlled by the surface properties of the biomaterial, the form of the implant, and the relationship between the surface area of the biomaterial and the volume of the implant. For example, high surface-to-volume implants such as fabrics or porous materials will have higher ratios of macrophages and foreign body giant cells in the implant site than smoother surface implants, which will have fibrosis (fibrous encapsulation) as a significant component of the implant site. Generally, fibrosis surrounds the biomaterial or implant with its interfacial foreign body reaction, isolating the implant and the foreign body reaction from the local tissue environment and the rate of its degradation will be substantially decreased. In recent findings it has been suggested that the modulus of a material is important for encapsulation and it has been proposed that a new material should have a modulus close to the surrounding tissue in order to minimize the thickness of the encapsulation layer.

Transport of nutrients to and waste products from the cell is critical for the cell's ability to proliferate and in a second step colonize an artificial scaffold in vivo. For chondrocytes this is accomplished by diffusion and for osteoblasts and most other cells this is achieved by in-growth of new blood vessels into the scaffold. Thus the material for bone regeneration should have an open and porous structure allowing angiogenesis.

By optimizing pore sizes, modulus and surface characteristics of the implant it would be possible to tailor materials that allow for in growth of cells, angiogenesis and that, at the same time, does not cause a to intense inflammatory reaction, which would be detrimental for the outcome of the surgical procedure

From an orthopaedic point of view the materials can usefully be divided in two segments; a first, where the focus is on materials that can stimulate growth of new bone but were physical strength is not necessary of importance. A second, which focus on weight bearing properties and mechanical strength and where the role of the implanted bone substitute is to stabilize a fracture or defect and mobilize the patient as soon as possible.

Several approaches have been tested but with limited success so far. The synthetic materials have either poor handling characteristics, to hard or to brittle, or cause unwanted side effects, following upon degradation of the materials. These shortcomings are reflected in the current US market figures for bone replacement materials (2001). The total market is, 578 million USD of which 96% (552 MUSD) comes from allografts and the remaining 4% (26 MSUD) comes from synthetic bone replacement materials. There therefore remains a need in the art for materials with improved handling and stability characteristics.

Accordingly, the present invention provides an orthopaedic composition comprising porous chitosan particles suspended in a liquid medium wherein the liquid medium further comprises a biocompatible polymer.

The invention addresses the problems associated with chitosan materials and their use e.g. in orthopaedic applications. The new chitosan materials of the present invention allow for high loadability, desired elastic properties, good cell adhesion and cell proliferation. These materials can be made to exhibit various pore characteristics and can be made from super-saturated chitosan mixtures of which at least one part is in the form of solid material. The materials can be characterized to comprise solid particles bound together in a matrix created from a liquid or gel formulation with subsequent drying of the resulting paste to generate the final materials.

In one embodiment the solid particles can be made porous before they are bound together, yielding a double or multi porous material, e.g. with pores of one size distribution within the particles and pores of a different size distribution between the particles. By using the methods disclosed in the present patent application, materials can be tailored to be designed for various uses, e.g. as bone filling or bone fixation devices. Materials intended for bone filling are softer but still have some load-bearing properties whereas materials intended for fixation are even stronger and can be shaped in commonly used forms, e.g. plugs, screws, plates etc. To increase further the rigidity of the particles, cross-linking can be used, either ionically or covalently.

According to one embodiment of the invention the double or multi porous material may be used as a coating material for medical devices made of e.g. stainless steel or titanium. Another object of the invention is to provide materials with physical properties similar to those of natural bone or tissue, i.e. loadability and flexibility. Another object of the invention is to provide porous materials that stimulate and support new bone growth. Another object of the invention is to provide materials in which pore sizes can be controlled in order to give optimized properties, e.g. biological properties like inflammation, encapsulation and other biological reactions. Another object of the invention is to provide double or multi porous materials with pores within the particles as well as in the matrix between the particles. Another object of the invention is to provide materials having controlled biodegradability, e.g. using chitosans of different degrees of N-deacetylation or mixtures of said chitosans. Alternatively, biodegradability can be affected by additional components included in the matrix structure, e.g. by adding polymers of different degradation rate. Another object of the invention is to provide materials that give non-toxic degradation products. Another object of the invention is to provide materials that can be given additional properties by incorporation of other biologically active molecules, e.g. growth factors, growth factor stimulating agents, anti-microbial agents, gene fragments, vitamins, pain relieving drugs etc. Another object of the invention is to provide a material that has inherent anti-microbial properties. Another object of the invention is to provide a material that is easy to handle. Another object of the invention is to provide a material that can be made in attractive physical forms and shapes for various uses. Another object of the invention is to provide a material that does not transmit diseases. Another object of the invention is to provide a material that can be used as bone chips. Another object of the invention is to provide a material that can be used for making bone wedges and bone plugs. Another object of the invention is to provide a material that can be used for cartilage repair. Another object of the invention is to provide a material that allows for angiogenesis. Another object of the invention is to provide a material that may be pre-seeded with living cells.

The present invention will now be described with reference to the accompanying drawings in which:

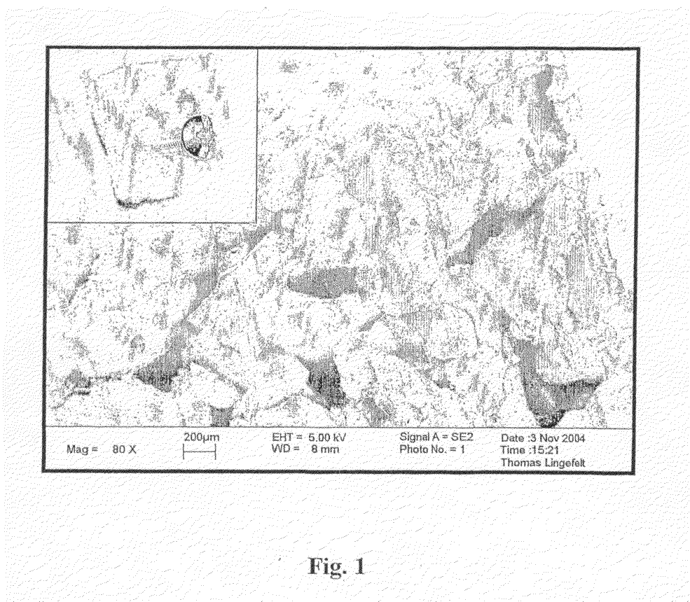

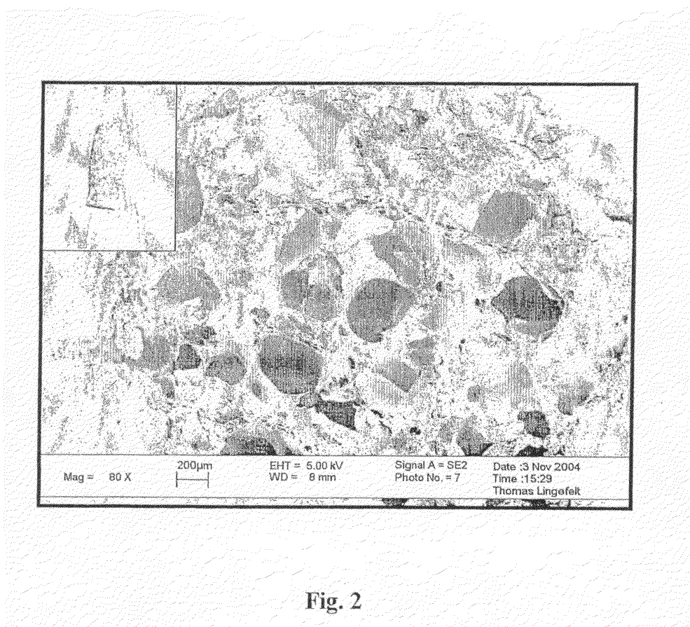

FIGS. 1 and 2 show materials formed by air-drying a composition of the present invention;

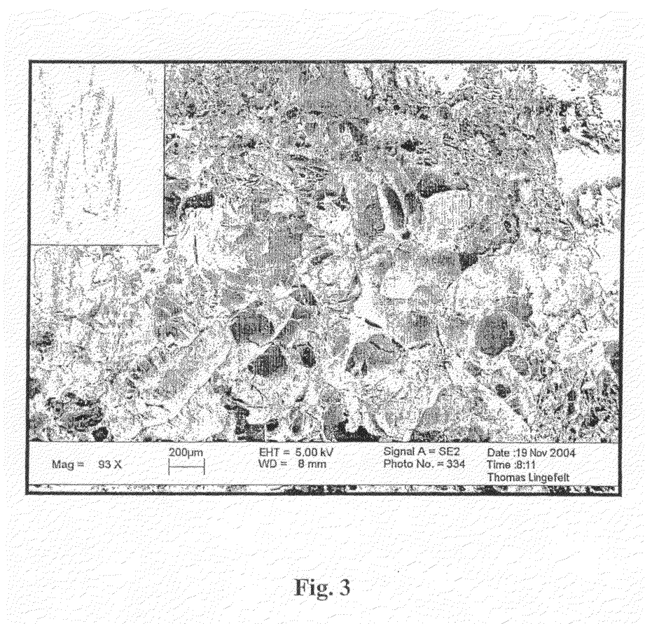

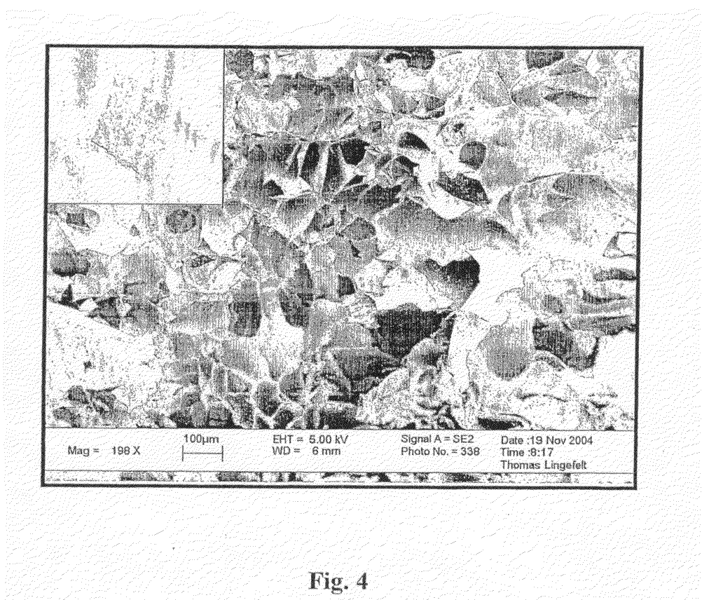

FIGS. 3 and 4 show freeze-dried materials;

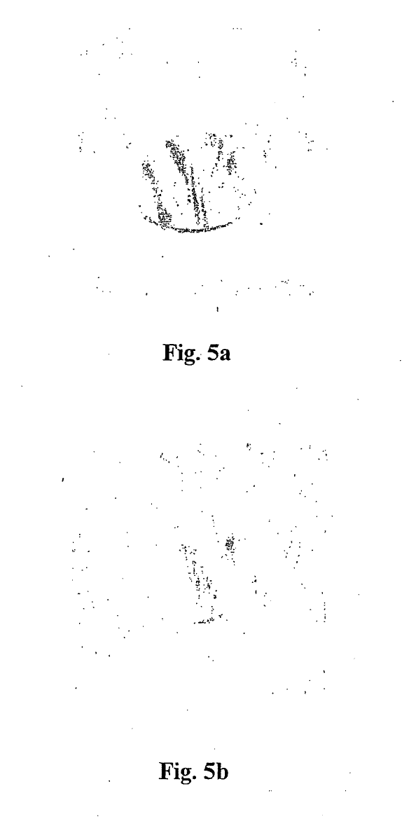

FIGS. 5a and b show the same material which has been dried (a) by freeze drying and (b) by air drying and

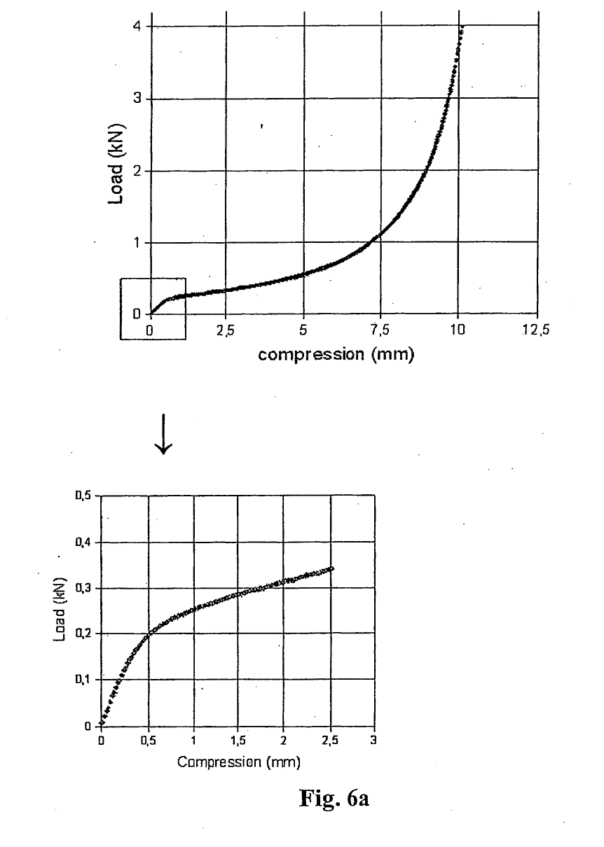

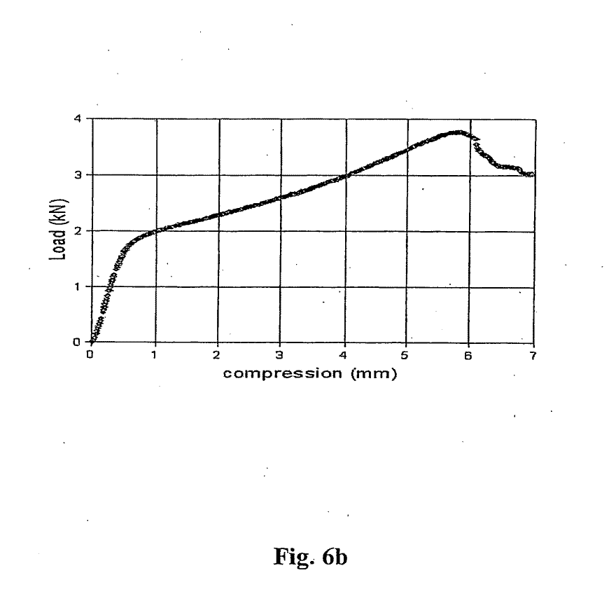

FIGS. 6a and 6b show compression data for (a) a freeze-dried material and (b) an air-dried material.

The present invention relates in general to materials made from chitosan intended e.g. for use in human and veterinary medicine. More specifically the present invention is aiming for products within the orthopaedic area, especially products used for healing of fractures, healing of cartilaginous tissue and bone defects or dental surgery. The products may also be used in cosmetic or plastic surgery.

Chitin is next to cellulose the most abundant polysaccharide on earth. It is found in hard structures and strong materials in which it has a function of a reinforcement bar. Together with calcium salts, some proteins and lipids it builds up the exoskeletons of marine organisms like crustaceans and arthropods. It is also found in the cell walls of some bacteria and sponges and build up the hard shells and wings of insects. Commercially, chitin is isolated from crustacean shells, which is a waste product from the fish industry. Chitosan is a linear polysaccharide composed of 1,4-beta-linked D-glucosamine and N-acetyl-D-glucosamine residues. Chitin in it self is not water soluble, which strongly limits its use. However, treatment of chitin with strong alkali gives the partly deacetylated and water-soluble derivative chitosan which can be processed in a number of different physical forms, e.g. films, sponges, beads, hydrogels, membranes. Chitosans in their base form, and in particular those of high molecular weight, and/or high degrees of N-deacetylation, are practically insoluble in water, however its salt with monobasic acids tend to be water-soluble. The average pKa of the glucosamine residues is about 6.8 and the polymer forms water-soluble salts with e.g. HCl, acetic acid, and glycolic acid.

Chitosan used in the present invention may be any deacetylated chitosan. However the chitosan preferably has a degree of deacetylation at least 33%, more preferably at least 40% and most preferably at least 50%; and preferably 100% or less, more preferably 95% or less and most preferably 90% or less. In general the lower the degree of deacetylation the more rapidly the chitosan will degrade when in contact with bodily fluids. The chitosan can be of pharmaceutical grade or equivalent quality e.g. the Chitech® quality provided by Carmeda AB, Sweden. The chitosan should not contain excessive levels of heavy metals, proteins, endotoxins or other potentially toxic contaminants. In many applications the chitosan should be essentially free from such compounds. The chitosan used in the porous chitosan particles and as the biocompatible polymer may have different degrees of deacetylation.

The chitosan is not specifically restricted in molecular weight. However, it preferably has a molecular weight of at least 5 kD, more preferably at least 10 kD and most preferably at least 15 kD; preferably 1500 kD or less, more preferably 1000 kD or less and most preferably 500 kD or less. The chitosan used in the porous chitosan particles and as the biocompatible polymer may have different molecular weights.

Like chitin, chitosan is a very strong polymer and it is also has several biological attractive properties. In-vivo degradation of chitosan occurs by enzymatic cleavage of the polymer chain. Lysozyme, which is found in almost all body fluids, is the most prominent of the chitosan degrading enzymes. A prerequisite for lysozyme to cleave is that there are remaining acetyl groups on the polysaccharide chain, and the more acetyl groups the faster is the degradation rate. Chitosan degrades to non-toxic components, it sticks to living tissue and it has antibacterial properties. These properties have made it very attractive in the development of medicinal products. It is used in e.g. products for control release of drugs, matrixes for cell cultivation, carriers for vaccines and products for wound healing, just to mention a few. The good biocompatibility of chitosan has been demonstrated in several in vivo studies and it has also been shown that bone cells, osteoblasts, can be cultured on matrixes built from chitosan. The potential of chitosan in orthopaedic applications has been postulated for long, its biological and physical properties are striking but up to now no one has been able to make materials strong enough to be used as substitute for skeleton or for bone fixation devices.

Chitosan may also be used mixtures of chitosans of different degree of N-deacetylation. Derivatives of chitosan in which the repeating units are substituted with biocompatible substituents may also be used. Examples of chitosan derivatives are sulphated chitosan, N-carboxymethyl chitosan, O-carboxymethyl chitosan and N,O-carboxymethyl chitosan.

The orthopaedic composition of the present invention is made from particles comprising chitosan suspended in a liquid medium. The liquid medium is therefore sufficiently viscous to maintain the chitosan particles in suspension, i.e. without settling of the chitosan particles. Such a medium is typically termed a “gel” in the art. This is achieved by incorporating a biocompatible polymer in the liquid phase. Preferably the biocompatible polymer is a polysaccharide or protein. Examples include chitosan and derivatives thereof, cellulose and derivatives thereof, hyaluronic acid, dextran chonroitin sulphate, heparin, alginic acid, collagen, fibrin, tissue sealants. The biocompatible polymer may be a charged (cationic or anionic) polymer or a non-charged polymer. More preferably the biocompatible polymer is a cationic polymer and most preferably chitosan or derivatives thereof. The biocompatible polymer may be dissolved or suspended in the liquid medium and typically forms a gel. The liquid medium is preferably water.

Although the viscosity varies with the nature of the composition, preferably the viscosity is at least 50 mPas, more preferably at least 100 mPas, more preferably at least 250 mPas, more preferably at least 500 mPas, more preferably at least 1000 mPas and most preferably at least 1500 mPas. The upper limit of viscosity is limited only by the handling requirements of the composition.

The amount of biocompatible polymer present will depend on the nature of the polymer since the nature of the polymer will determine the viscosity increase in the liquid medium. The viscosity required will also depend on the size and nature of the porous chitosan particles since different particles will require a different viscosity to enable the particles to remain in solution. However, the amount of biocompatible polymer will typically be at least 0.1%, more preferably at least 1%; and no more than 20%, more preferably no more than 15%, more preferably no more than 10%, and most preferably no more than 5% by weight, based on the total weight of the liquid medium (i.e. not including the porous chitosan particles). Preferably the liquid medium is supersaturated with the biocompatible polymer. Where the biocompatible polymer is chitosan, when making gels and water solutions in an acidic environment there is a practical limit set by the solubility of the specific chitosan, which is dependent on its molecular weight and its degree of N-deacetylation. However, the amount of chitosan in an aqueous medium is typically in a range from 1-10%, preferably 1-5% by weight based on the weight of the liquid medium, with the amount tending towards the higher end of the range if low molecular weight chitosans are used.

Said suspensions or pastes can be used as such, but are most often shaped into desired forms and dried. Thus, the present invention provides a process for preparing a solid or semi-solid orthopaedic material comprising drying the orthopaedic composition as described herein. By semi-solid is meant a material which is not completely dried to form a solid. Drying can be performed for example by evaporation of the liquid medium, e.g. by air drying or drying under reduced pressure, or by freeze-drying to give the desired materials. The present invention also provides a solid or semi-solid orthopaedic material which may be obtained by this process. The drying conditions have great influence on the matrix created by the paste material where the particles of the composition are bound more or less close to each other. Air drying results in a more dense material with smaller pores resulting in a material of higher mechanical strength. Freeze drying introduces larger pores into the matrix between the individual porous chitosan particles thereby providing a material which is less strong but has greater flexibility and which is suitable for e.g. in-growth of cells and blood vessels. The pores produced by freeze drying have a diameter from about 50 μm to several millimetres (up to around 1 cm) and the pores produced by air drying have a diameter of about 50 and 200 μm. In particular this offers a possibility to achieve a desired matrix porosity, in addition to the porosity of the porous chitosan particles in the paste, allowing the properties of dried material to be tailored to particular applications.

In addition the properties of the materials can be altered by addition of additives commonly used in pharmaceutical compositions, e.g. preservatives, lubricants or plasticisers, e.g. glycerol. Plasticisers such as glycerol tend to increase the flexibility of the dried material and may be used to give a soft, malleable paste that may be used for the filling of bone defects

These dried materials can be further processed or sculptured e.g. threaded or drilled, or milled into flakes. This paste can also be applied to the surface of other materials, e.g. stainless steel or titanium to give a rough semi-solid, anti-microbial protection. Some of these properties may be seen in the figures.

FIG. 1 shows a dried material in the form of a plate having a screw screwed into the plate. The plate was prepared by air-drying and a composition having a small quantity of glycerol added thereto as set out in Example 1 hereinbelow. The dense microstructure of the plate may also be seen from the photomicrograph.

FIG. 2 also shows an air-dried material. The shaped bar contains a screw thread on its external surface.

FIGS. 3 and 4 show freeze-dried materials. The macropores are obtained by the removal of water in the freeze drying process. The water leaves but the three-dimensional structure remains providing a more porous but less strong material.

FIGS. 5a and b show the same material which has been dried in a different manner, as set out in Examples 4:8 and 4:9 hereinbelow. The plug in FIG. 5a was freeze dried (lyophilised) and has a diameter of 12 mm and a length of 13 mm. The plug in FIG. 5b was air dried and has a diameter of 7 mm and a length of 13 mm.

Particles comprising chitosan can be made in many ways. One way is by milling of the solid residue obtained from evaporation of a chitosan solution. Another is to mill chitosan fibres or the chitosan flakes, which is the product in most chitosan processes. Porous chitosan particles and beads can be prepared by using cross-linking agents like polyphosphates or from detergent containing solutions. Another way of generating pores in a chitosan material is to use porogens. In general porogens are molecules added to give a material a specific structure during its formation and which can subsequently be removed, e.g. by washing. Typical porogens are oligosaccharides, low molecular weight polyethylene glycols, glycerol etc.

Another way of introducing large pores into a chitosan material is to use particles like silica particles as porogens. These are in a second step removed by washing with alkali solutions.

Surprisingly, it has been found that if e.g. an inorganic salt e.g. sodium chloride, potassium chloride, calcium chloride, and magnesium chloride, and most preferably sodium chloride, or a polyethylene glycol of high molecular weight (e.g. Mw at least 10 kD and preferably 20 kD) is used as the porogen, the residue after evaporation is brittle and consequently easy to mill. Without wishing to be bound by theory, it is believed that this is due to a “salt effect”. It has been found that even if other molecules, e.g. other glycosaminoglycans (GAGs), growth factors, proteins are added to some extent to the porogens-containing chitosan paste, the materials can still be milled after evaporation of the liquids. The ratio between the chitosan and the porogen can be from 1:1 to 1:10 and more preferably in the range from 1:2 to 1:5, depending on the desired porosity. This in contrast to previously known materials, such as those disclosed in WO 01/46266 and Macromol. Biosci. 2004, 4, 811-819, discussed hereinabove, which cannot be milled since they tend to agglomerate leading to undesired heating which can chemically degrade the chitosan.

Accordingly the present invention also provides a process for preparing porous chitosan particles comprising: preparing a solution containing chitosan and a porogen capable of inducing crystallinity in to the chitosan, drying the solution to a solid residue, and milling the solid residue to generate the porous chitosan particles. The present invention also provides porous chitosan particles obtainable by this process. Such particles are particularly preferred particles for incorporation into the orthopaedic composition of the present invention.

The porogens may then be removed, e.g. by neutralisation of the particles containing porogens, in an alkaline buffer, with subsequent extensive washing. Finally, porous particles are obtained by drying. If needed, these particles are then further fractioned, e.g. by sieving, to give particles of different sizes or a desired size for a specific application.

The porosity of the porous chitosan particles of the present invention increases which increasing amounts of porogen. This may be seen by viewing the particle with an electron microscope and analysing the percentage pore volume compared to the total cross-sectional area of the particle. A 1:1 ratio of chitosan to sodium chloride provides a calculated % pore volume of 44.7. A similar calculation for a ratio of chitosan to salt of 1:2, 1:3, 1:4, 1:5 and 1:10 gives a pore volume of 62%, 71%, 78%, 80% and 89%, respectively. Preferably the porous chitosan particle has a % pore volume of at least 40%, more preferably at least 60%, more preferably at least 65% and most preferably at least 70%; and no more than 95%, more preferably no more than 90%, more preferably no more than 85% and most preferably no more than 80%.

The chitosan particles may contain other materials in addition to chitosan, although some chitosan must be present. Preferably the particles contain at least 50% chitosan, and more preferably 50 to 90% chitosan. The remainder of the particles may include derivatives of chitosan, and/or other polysaccharides and/or proteins. The chitosan particles may be used in combination with other particulate materials, e.g. a drug containing granulate for slow or controlled release of a desired compound, e.g. antibiotics, anti inflammatory or pain killing substances, or a particle containing molecules promoting cell growth, e.g. growth factors or molecules known to stabilize growth factors. When the milled material is used as bone chips it may be mixed with allograft bone chips in any ratio. Living bone forming cells, osteoblasts may be added to the bone chips. The products according to the invention may contain chitosan particles of different size, different pore size, different composition and/or different chitosan quality, e.g. chitosans of different degrees of deacetylation. The particles or the mixture of particles are then added to a gel or a solution in such a concentration that the solution becomes super-saturated with respect to chitosan, meaning that even if the solution is made acidic the chitosan still, at least to some extent, remains in particulate form. Acid treatment of the particles gives a protonated chitosan surface that is gel-like and sticky.

When preparing a chitosan solution intended for making particles the chitosan can be dissolved in an acidic environment, i.e. pH below 7. Preferred acids are acetic acid, hydrochloric acid and alpha-hydroxyacids, e.g. glycolic acid.

By combining the particles with the liquid medium described hereinabove, materials can be tailored to obtain desired properties and forms. This can be accomplished by using particles of different size and/or of different porosity. Other parameters affecting the properties of the materials will be the concentration of particles added to the paste and the way the paste is dried, as discussed hereinabove. Surprisingly it was found that by varying the above parameters bone-like materials could be produced.

Biologically active molecules, e.g. growth factors, growth factor stimulating agents, anti-microbial agents, gene fragments, vitamins, pain relieving drugs, etc, may be added alone or in mixtures when preparing the particles, the liquid medium or both. Examples of such biologically active molecules are bone morphogenic proteins e.g. recombinant human bone morphogenic protein-2 (rhBMP-2) or recombinant human bone morphogenic protein-7 (rhBMP-7), fibroblast growth factors (FGF), platelet derived growth factor (PDGF), transforming growth factor-b, growth hormone and insulin like growth factors, gentamicin, rifampin, flucloxacillin, vancomycin, ciprofloxacin, ofloxacin, penicillin, cephalosporin, griseofulvin, bacitracin, polymyxin B, amphotericin B, erythromycin, neomycin, streptomycin, tetracycline, salicylates, ibuprofen, naproxen, morphine, meperidine, propoxyphen, diclofenac, diflunical, etodolac, fenoprofen, indomethacin, ketoprofen, ketorolac, meclofenamate, metenamic acid, ecopan, oxaproein, sulindac, tolmetin, vitamin A, vitamin B, vitamin C, vitamin D, vitamin E, vitamin K.

Living cells may also be added to the material according to the invention. Examples of such cells are osteoblasts and chondrocytes.

The material according to the invention can be tailored to meet any need. By varying the pore size of the particles the physical properties may be tailored. Larger pores give a softer more elastic material whereas small pores give a harder material. Biologically active molecules may be incorporated in the porous particles to give a slow release of these molecules as the material degrades. The pore size may further be varied in order to obtain a material that is a suitable matrix for the culture of bone and cartilage forming cells. Chitosan in itself stimulates osteoblast and chondrocyte growth and by producing particles of an optimal pore size the material according the invention becomes an optimal scaffold for cell culture. The gel may contain chitosan of another, e.g. lower, degree of N-deacetylation than the particles so that the degradation rate of the gel is faster than that of the particles. Such a material is strong initially but degrades after a period of time to leave only the particles which are readily accessible to in-growing cells.

One example of a product according to the invention is the chitosan particle-containing paste as disclosed above, which can be distributed for local use where it is allowed to dry or substantially dry to a body of a desired shape. Another example of a product is the dry material obtained by a drying process.

The dry materials according to the invention swell in aqueous solutions and the degree of swelling can be tailored to meet the requirements for different uses by e.g. varying the degree of deacetylation of the chitosan(s) used.

Accordingly, the solid or semi-solid orthopaedic material of the present invention finds use as a bone-replacement material, a bone cement and a tissue scaffold. The solid orthopaedic material may also be fabricated to form materials for osteosynthesis, such as screws, pins, plates, pegs, rivets, cotters, spikes, bolts, studs, staples, bosses, clamps, clips, dowels, stakes, hooks, anchors, ties, bands, crimps, wedges, plugs, nails, wires, rings, ring fixators, and washers.

The invention is illustrated, but in no way limited, by the following examples.

### EXAMPLES

The following materials were used in the Examples unless otherwise stated:

Chitosan from Primex, Norway, 145 kD and 85% degree of N-deacetylation. Chitosans of lower degree of N-deacetylation were prepared essentially following the principles outlined in: Sannan T, Kurita K, Iwakura Y. Studies on Chitin, 1. Die Makromolekulare Chemie 1975; 0:1191-5, Sannan T, Kurita K, Iwakura Y. Studies on Chitin, 2. Makromol. Chem. 1976; 0:3589-600, Guo X, Kikuch, Matahira Y, Sakai K, Ogawa K. Water soluble Chitin of low degree of deacetylation. Journal of Carbohydrate chemistry 2002; 21:149-61 and WO03011912. Hyaluronic acid from Pharmacia, Glycerol from Fluka, Germany, NaCl from Merck, MgCl₂ from Merck, HCl from Merck, Water millipore

### Example 1

4 g chitosan (degree of N-deacetylation 85%, MW 145 kD) was dissolved in 133 g water by adjusting the pH to 4.5 with diluted HCl. To the stirred chitosan solution was then added an aqueous solution of 12 g NaCl dissolved in 50 g water. The gel like slurry was then spread out on a flat plastic surface and air-dried to give a brittle residue which was further grinded into particles (250 μm). Subsequent neutralisation of the particles in an alkaline buffer and extensive washing with water gave a porous chitosan matrix free of salt. After drying, 0.3 g of the porous particles were added to a gel (1.2 g) consisting of 4% chitosan (degree of N-deacetylation 85%, 145 kD) pH 4.5, and 0.4 g glycerol. The paste was swelled for 2 minutes at room temperature, spread on a plastic surface and shaped into a plate (20×20×2 mm). After drying at 40° C. a strong slightly flexible plate was obtained.

### Example 2

3 g chitosan (degree of N-deacetylation 50%, MW 200 kD) was dissolved in 134 g water by adjusting the pH to 4.5 with diluted HCl. To the stirred chitosan solution was then added 50 g of an aqueous solution of 12 g of NaCl and 0.3 g hyaluronic acid. The gel like slurry was spread out on a flat plastic surface and air-dried to dryness and milled into particles (250 μm). The particles were then neutralised, washed with water, dried and milled into particles. 0.3 g of the dried porous particles was then thoroughly mixed with 1.2 g of a 4% chitosan solution/gel of pH 4.5 (degree of N-deacetylation 50% MW 200 kD) to give a paste. The paste was allowed swelling for 2 minutes at room temperature and shaped into rods by moulding the paste into tubes. Freeze drying of the filled tubes and subsequent removal of the tube gave strong porous rods containing chitosan/hyaluronic acid complexes.

### Example 3

4 g chitosan (degree of N-deacetylation 85%, MW 145 kD) was dissolved in 133 g water by adjusting the pH to 4.5 with diluted HCl. To the chitosan solution was then added 20 g MgCl₂ dissolved in 43 g water. The gel like slurry was spread out on a flat plastic surface air-dried and milled into particles (1 mm). The particles/flakes were neutralised in an alkaline buffer, washed with water and dried. 0.3 g of the dried porous particles were then added to 11.0 g of 4% chitosan solution/gel of pH 4.5 (degree of N-deacetylation 85%, MW 145 kD) and thoroughly mixed to a paste which was allowed to swell for 2 minutes at room temperature. After swelling the paste was shaped into plugs by moulding the paste into short tubes (Ø=5 mm, h=10 mm). Freeze drying of the tubes and removal of the tubes gave strong chitosan plugs.

Other materials have been manufactured according to the similar procedures in which the size and concentration of particles and the drying procedure have been varied.

### Example 4

### Preparation of Chitosan Particles

18.31 g of chitosan (degree of N-deacetylation 85%, MW 145 kD) was dissolved in 570 g water by adjusting the pH to 4.5 with diluted HCl. The weight was adjusted to 600 g with water.

54 g of NaCl was dissolved in 171 g water.

### Example 4:1

### Preparation of Chitosan Particles

150 g of the chitosan solution above was added to 37.5 g of the NaCl-solution above and 37.5 g of water was added. The mixture was stirred until it became homogeneous. The mixture was spread out on a flat plastic surface and was air-dried. The dry flakes obtained were milled using a Retsch ZM 200 mill, equipped with a 250 μm ring sieve, at 14000 rpm. The particles were neutralised with a iN NaOH solution, washed with water (5×300 ml) and air-dried.

### Example 4:2

### Preparation of Chitosan Particles

450 g of the chitosan solution above was added to 168.8 g of the NaCI-solution above. The mixture was stirred until it became homogeneous. The mixture was spread out on a flat plastic surface and was air-dried. The dry flakes obtained were milled using a Retsch ZM 200 mill, equipped with a 80, 120 and 250 μm ring sieve, respectively, at 14000 rpm. The particles were neutralised with a IN NaOH solution, washed with water and air-dried.

### Example 4:3

### Preparation of Chitosan Particles

150 g of the chitosan solution above was added to 75.0 g of the NaCl-solution above and 37.5 g of water was added. The mixture was stirred until it became homogeneous. The mixture was spread out on a flat plastic surface and was air-dried. The dry flakes obtained were milled using a Retsch ZM 200 mill, equipped with a 250 μm ring sieve, at 14000 rpm. The particles were neutralised with a 1N NaOH solution, washed with water and air-dried.

### Example 4:4

### Preparation of Chitosan Gels

21.0 g of chitosan (degree of N-deacetylation 85%, MW 145 kD) was dissolved in 650 g of water. The pH was adjusted to 3.5 with 4N HCI. The weight was adjusted to 700 g, to give a 3% chitosan solution.

### Example 4:5

### Preparation of Chitosan Gels

25.0 g of chitosan (degree of N-deacetylation 85%, MW 145 kD) was dissolved in 450 g of water. The pH was adjusted to 5.1 with 4N HCl. The weight was adjusted to 500 g, to give a 5% chitosan solution.

### Example 4:6

### Preparation of Chitosan Plugs

3 g of the 80 μm chitosan particles of Example 4: 2 were mixed with 15 g of the 5% chitosan gel of Example 4: 5. The paste formed was placed in cylindrical moulds with a diameter of 13 mm and the samples were air-dried or lyophilised, to give a solid material which was further mechanically processed to give chitosan plugs of the typical sizes shown in Tables 2 and 3.

### Example 4:7-4.22

According to the above procedure chitosan plugs were prepared as summarised in Table 1.

TABLE 1

| | | Particle | | Weight | | |
|---|---|---|---|---|---|---|
| | Chitosan | size | Chitosan | ratio | Drying | Glycerol |
| Sample no | particles | (μm) | gel | particles:gel | method | (% w/w) |
| 4:6 | Ex. 4:2 | 80 | Ex. 4:5 | 1:5 | Air-dried | |
| 4:7 | Ex. 4:2 | 80 | Ex. 4:5 | 1:5 | Lyophilised | |
| 4:8 | Ex. 4:2 | 120 | Ex. 4:5 | 1:5 | Air-dried | |
| 4:9 | Ex. 4:2 | 120 | Ex. 4:5 | 1:5 | Lyophilised | |
| 4:10 | Ex. 4:2 | 250 | Ex. 4:5 | 1:10 | Air-dried | |
| 4:11 | Ex. 4:2 | 250 | Ex. 4:5 | 1:10 | Lyophilised | |
| 4:12 | Ex. 4:2 | 250 | Ex. 4:5 | 1:5 | Air-dried | |
| 4:13 | Ex. 4:2 | 250 | Ex. 4:5 | 1:5 | Lyophilised | |
| 4:14 | Ex. 4:2 | 250 | Ex. 4:5 | 1:5 | Air-dried | 17% |
| 4:15 | Ex. 4:2 | 250 | Ex. 4:5 | 1:5 | Lyophilised | 17% |
| 4:16 | Ex. 4:2 | 250 | Ex. 4:4 | 1:5 | Air-dried | |
| 4:17 | Ex. 4:2 | 250 | Ex. 4:4 | 1:5 | Lyophilised | |
| 4:18 | Ex. 4:1 | 250 | Ex. 4:5 | 1:5 | Air-dried | |
| 4:19 | Ex. 4:1 | 250 | Ex. 4:5 | 1:5 | Lyophilised | |
| 4:20 | Ex. 4:3 | 250 | Ex. 4:5 | 1:5 | Air-dried | |
| 4:21 | Ex. 4:3 | 250 | Ex. 4:5 | 1:5 | Lyophilised | |
| 4:22 | No | — | Ex. 4:5 | | Lyophilised | |
| | particles | | | | | |

### Example 5

The chitosan plugs prepared according to Example 4 were tested for break points and compressive modulus. Compressive data and break points for the lyophilised and air-dried plugs was measured on a Sintech 20 D apparatus equipped with a 10 kN load cell operating at a compression rate of 1 mm/min. Plug sizes are given in the tables. The data for lyophilised plugs is given in Table 2 and FIG. 6a and for air-dried plugs in Table 3 and FIG. 6b. FIG. 6a shows graphically the compression analysis for sample 4:7 and FIG. 6b shows graphically the compression analysis for sample 4:6. The lyophilised plugs and the air-dried plugs containing glycerol had no break points and hence only compressive modulus data are given.

TABLE 2

| | | | Compressive |
|---|---|---|---|
| Sample no. | Diameter | Length | Modulus (MPa) |
| 4:7 | 11.8 | 13.1 | 50.49 |
| 4:7 | 11.7 | 12.5 | 44.33 |
| 4:9 | 11.8 | 11.2 | 33.99 |
| 4:9 | 11.9 | 12.5 | 46.17 |
| 4:11 | 11.8 | 12.5 | 19.85 |
| 4:11 | 11.7 | 12.0 | 16.1 |
| 4:13 | 11.9 | 12.1 | 16.1 |
| 4:13 | 11.8 | 11.1 | 36.38 |
| 4:15 | 11.5 | 12.8 | 20.87 |
| 4:15 | 11.6 | 12.0 | 36.46 |
| 4:17 | 11.7 | 11.3 | 13.45 |
| 4:17 | 11.9 | 12.5 | 12.38 |
| 4:19 | 11.9 | 12.5 | 41.59 |
| 4:19 | 11.9 | 11.2 | 23.47 |
| 4:21 | 11.9 | 12.4 | 39.23 |
| 4:21 | 11.8 | 11.9 | 42.1 |
| 4:22 | 10.6 | 12.0 | 1.55 |
| 4:22 | 11.0 | 12.0 | 4.34 |

Samples 4:7 to 4:21 gave a higher compression modulus than sample 4:22 which does not contain any chitosan particles. Indeed, the dried materials of the present invention have a compressive modulus which is at least 100% higher than that obtained from a dried material identical in all respects other than that it is does not contain porous chitosan particles.

TABLE 3

| | | | Compressive | Load at Break |
|---|---|---|---|---|
| Sample no. | Diameter | Length | Modulus (MPa) | (kN) |
| 4:6 | 7.3 | 13.7 | 1084.67 | 3.01 |
| 4:6 | 7.3 | 12.7 | 1112.96 | 3.77 |
| 4:6 | 7.4 | 13.4 | 919.59 | 2.68 |
| 4:8 | 7.2 | 13.3 | 1309.44 | 3.42 |
| 4:8 | 7.4 | 12.9 | 997.23 | 3.66 |
| 4:8 | 7.4 | 13.4 | 857.97 | 2.92 |
| 4:10 | 6.9 | 12.7 | 453.58 | 0.51 |
| 4:12 | 7.65 | 12.7 | 1241.16 | 2.69 |
| 4:12 | 7.2 | 12.6 | 1056.06 | 0.85 |
| 4:12 | 7.8 | 13.9 | 815.56 | 1.14 |
| 4:14 | 7.7 | 13.0 | 418.75 | |
| 4:14 | 8.0 | 13.3 | 329.93 | |
| 4:14 | 8.0 | 13.7 | 217.21 | |
| 4:16 | 7.7 | 13.7 | 685.04 | 0.63 |
| 4:16 | 7.6 | 12.7 | 1092.27 | 1.15 |
| 4:16 | 6.9 | 13.5 | 868.58 | 0.93 |
| 4:18 | 7.5 | 13.4 | 1019 | 4.84 |
| 4:18 | 7.5 | 13.5 | 907.7 | 4.00 |
| 4:20 | 7.5 | 13.9 | 947.37 | 1.76 |
| 4:20 | 7.4 | 12.3 | 794.87 | 1.48 |
| 4:20 | 7.7 | 13.7 | 527.8 | 1.29 |

1. An orthopaedic composition comprising porous chitosan particles suspended in a liquid medium wherein the liquid medium further comprises a biocompatible polymer.

2. An orthopaedic composition as claimed in claim 1, wherein the particles have a particle size of 10 μm to 2 mm.

3. An orthopaedic composition as claimed in claim 1 or 2, wherein the particles are composed of a mixture of chitosan together with derivatives of chitosan, polysaccharides and/or proteins.

4. An orthopaedic composition as claimed in claim 3, wherein the derivatives are selected from sulphated chitosan, N-carboxymethyl chitosan, O-carboxymethyl chitosan and N,O-carboxymethyl chitosan

5. An orthopaedic composition as claimed in any preceding claim, wherein the particles contain at least 50% chitosan.

6. An orthopaedic composition as claimed in any preceding claim, wherein the particles contain 50 to 90% chitosan.

7. An orthopaedic composition as claimed in any preceding claim, wherein the liquid medium further comprises a plasticiser.

8. An orthopaedic composition as claimed in claim 7, wherein the plasticiser is glycerol.

9. An orthopaedic composition as claimed in any preceding claim, wherein the biocompatible polymer is a polysaccharide or protein.

10. An orthopaedic composition as claimed in any preceding claim, wherein the biocompatible polymer is a charged polymer.

11. An orthopaedic composition as claimed in claim 10, wherein the biocompatible polymer is a cationic polymer.

12. An orthopaedic composition as claimed in claim 11, wherein the biocompatible polymer is chitosan.

13. An orthopaedic composition as claimed in any preceding claim, wherein the biocompatible polymer is dissolved in the liquid medium.

14. An orthopaedic composition as claimed in any preceding claim, wherein the liquid medium is an aqueous medium.

15. A process for preparing a solid or semi-solid orthopaedic material comprising drying the orthopaedic composition as claimed in any preceding claim.

16. A process for preparing a solid or semi-solid orthopaedic material as claimed in claim 15, wherein drying is carried out by freeze drying.

17. A process for preparing a solid or semi-solid orthopaedic material as claimed in claim 15, wherein drying is carried out by evaporation of the liquid medium.

18. A solid or semi-solid orthopaedic material obtainable by the process as claimed in any of claims 15 to 17.

19. A solid or semi-solid orthopaedic material as claimed in claim 18, wherein the material contains pores between the porous chitosan particles having a diameter of 50 μm to 1 cm.

20. Use of the solid or semi-solid orthopaedic material as claimed in claims 18 or 19 as a bone-replacement material.

21. Use of the solid or semi-solid orthopaedic material as claimed in claims 18 or 19 as a bone cement.

22. Use of the solid or semi-solid orthopaedic material as claimed in claims 18 or 19 as a tissue scaffold.

23. A process for preparing porous chitosan particles comprising: preparing a solution containing chitosan and a porogen capable of inducing crystallinity in to the chitosan, drying the solution to a solid residue, and milling the solid residue to generate the porous chitosan particles.

24. A process as claimed in claim 23, wherein the solution contains of a mixture of chitosan together with derivatives of chitosan, polysaccharides and/or proteins.

25. A process as claimed in claim 23 or 24, wherein the derivatives are selected from sulphated chitosan, N-carboxymethyl chitosan, O-carboxymethyl chitosan and N,O-carboxymethyl chitosan

26. A process as claimed in claims 24 or 25, wherein the mixture contains at least 50% chitosan.

27. A process as claimed in claim 26, wherein the mixture contains 50 to 90% chitosan.

28. A process as claimed in any of claims 23 to 27, wherein the porogen is selected from a biocompatible inorganic salt or a polyethylene glycol having a molecular weight of at least 10 kD.

29. A process as claimed in claim 28, wherein the porogen is a biocompatible inorganic salt and the salt is selected from sodium chloride, potassium chloride, calcium chloride, and magnesium chloride.

30. A process as claimed in claim 29, wherein the salt is sodium chloride.

31. A process as claimed in any of claims 23 to 30, wherein the ratio of chitosan or the mixture of chitosan together with derivatives of chitosan, polysaccharides and/or proteins to porogen is from 1:1 to 1:10.

32. A process as claimed in claim 31, wherein the ratio is from 1:2 to 1:5.

33. Porous chitosan particles obtainable by the process as claimed in any of claims 23 to 32.

34. An orthopaedic composition as claimed in any of claims 1 to 14, wherein the porous chitosan particle is the porous chitosan particle claimed in claim 33.
